(19) United States
(12) Patent Application Publication
(10) Pub. No.: US 2018/0214110 A1
(43) Pub. Date: Aug. 2, 2018

(54) MEDICAL IMAGE-PROCESSING APPARATUS, X-RAY CT APPARATUS, AND MEDICAL IMAGE-PROCESSING METHOD

(71) Applicant: Canon Medical Systems Corporation, Otawara-shi (JP)

(72) Inventor: Takuma IGARASHI, Nasushiobara (JP)

(73) Assignee: Canon Medical Systems Corporation, Otawara-shi (JP)

(21) Appl. No.: 15/886,287

(22) Filed: Feb. 1, 2018

(30) Foreign Application Priority Data
Feb. 2, 2017 (JP) 2017-017849

(51) Int. Cl. A61B 6/00; A61B 5/08; A61B 6/03; G06T 7/00; G06T 7/11; G06T 7/174; G06T 11/00

(52) U.S. Cl. CPC A61B 6/5217; A61B 6/50; A61B 5/0806; A61B 6/463; A61B 6/032; G06T 2207/30061; G06T 7/11; G06T 7/174; G06T 11/003; A61B 6/466; G06T 2207/10081; G06T 7/0016

(57) ABSTRACT

A medical image-processing apparatus of embodiments includes processing circuitry. The processing circuitry extracts plural regions corresponding to at least one of a lobe and a subsegment forming a lung from three-dimensional medical-image data that is acquired by imaging in temporal order. The processing circuitry calculates a physical index relating to respiratory activity for each of the extracted regions. The processing circuitry detects an abnormal region having an abnormality relating to the respiratory activity out of the regions by comparing temporal changes of the physical index of the respective regions. The processing circuitry outputs information indicating about the abnormal region.

### CROSS-REFERENCE TO RELATED APPLICATIONS

This application is based upon and claims the benefit of priority of the prior Japanese Patent Application No. 2017-017849, filed on Feb. 2, 2017, the entire contents of which are incorporated herein by reference. The entire contents of the prior Japanese Patent Application No. 2018-015731, filed on Jan. 31, 2018, are also incorporated herein by reference.

### FIELD

The embodiments described herein generally relate to a medical image-processing apparatus, an X-ray computerized-tomography (CT) apparatus, and a medical image-processing method.

### BACKGROUND

In pulmonary disease diagnosis, evaluation of respiratory activity has conventionally been practiced. For example, by observing a ventilation volume or a ventilation curve (spirogram) of an entire lung by using a spirometer, diagnosis for a chromic obstructive pulmonary disease (COPD) or the like has been done. However, for early detection of a pulmonary disease, such as COPD, spirometory is not sufficient for diagnosis in some cases. Moreover, it is difficult to find which part of a lung is impaired in its function by spirometory.

### BRIEF DESCRIPTION OF THE DRAWINGS

FIG. 1 is a diagram illustrating one example of a configuration of an X-ray CT apparatus according to a first embodiment;

FIG. 2A to FIG. 2E are diagrams for explaining processing of an extraction function according to the first embodiment;

FIG. 3A and FIG. 3B are diagrams for explaining processing of a detection function according to the first embodiment;

FIG. 4 is a diagram for explaining processing of an output control function according to the first embodiment;

FIG. 5 is a flowchart illustrating a procedure of processing performed by the X-ray CT apparatus according to the first embodiment;

FIG. 6 is a diagram for explaining processing of a detection function according to a modification of the first embodiment;

FIG. 7 is a diagram for explaining processing of a detection function according to a second embodiment;

FIG. 8A to FIG. 8D are diagrams for explaining processing of the detection function according to the second embodiment;

FIG. 9 is a diagram for explaining processing of an output control function according to the second embodiment;

FIG. 10 is a flowchart of a procedure of processing performed by an X-ray CT apparatus according to the second embodiment;

FIG. 11 is a diagram for explaining processing performed by an X-ray CT apparatus according to another embodiment;

FIG. 12 is a block diagram illustrating a configuration example of a medical image-processing apparatus according to another embodiment; and

FIG. 13 is a block diagram illustrating a configuration example of a server device that provides an information processing service according to another embodiment.

### DETAILED DESCRIPTION

A purpose to be achieved by an embodiment is to provide a medical image-processing apparatus, an X-ray CT apparatus, and a medical image-processing method that enables to detect a region of a lung at which the respiratory activity is abnormal accurately.

A medical image-processing apparatus of an embodiment includes processing circuitry. The processing circuitry extracts plural regions that correspond to at least one of a lobe and a subsegment forming a lung from three-dimensional medical-image data imaged temporally. The processing circuitry calculates a physical index relating to respiratory activity for each of the extracted regions. The processing circuitry detects an abnormal region relating to the respiratory activity out of the multiple regions by comparing a temporal change of the physical index of the respective regions. The processing circuitry outputs information that indicates about the abnormal region.

Embodiments of the medical image-processing apparatus and an X-ray CT apparatus are explained in detail below referring to the accompanying drawings. In the following embodiment, an X-ray CT apparatus that acquires X-ray CT image data of a subject is explained as an example. However, embodiments are not limited thereto, and are widely applicable to, for example, an X-ray diagnostic apparatus that can acquire three-dimensional X-ray image data, or a medical image-processing apparatus (computer) that can process three-dimensional medical-image data.

### First Embodiment

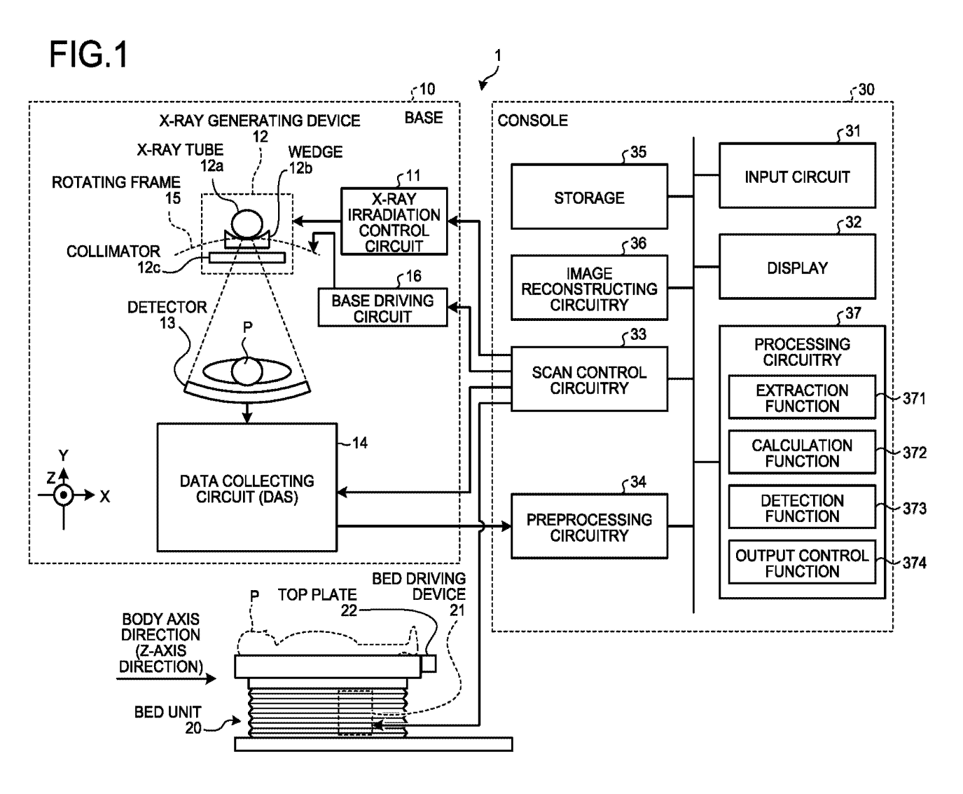

FIG. 1 illustrates one example of a configuration of an—X-ray CT apparatus 1 according to a first embodiment. As illustrated in FIG. 1, the X-ray CT apparatus 1 according to the first embodiment includes a base 10, a bed unit 20, and a console 30.

The base 10 is a device that irradiates an X-ray to a subject P (patient), and detects an X-ray that has passed through the subject P to output to the console 30, and includes an X-ray-irradiation control circuit 11, an X-ray generating device 12, a detector 13, a data collecting circuit (data acquisition system (DAS)) 14, a rotating frame 15, and a base driving circuit 16.

The rotating frame 15 is an annular-shaped frame that supports the X-ray generating device 12 and the detector 13 so as to oppose to each other about the subject P, and that rotates at high speed in a circular orbit about the subject P in center by the base driving circuit 16 described later.

The X-ray-irradiation control circuit 11 is a device that supplies a high voltage to an X-ray tube 12a as a high-voltage generating unit, and the X-ray tube 12a generates an X-ray by using the high voltage supplied from the X-ray-irradiation control circuit 11. The X-ray-irradiation control circuit 11 adjusts an amount of X-ray to be irradiated to the subject P by adjusting a tube voltage and a tube current to be supplied to the X-ray tube 12a under control of scan control circuitry 33 described later.

Moreover, the X-ray-irradiation control circuit 11 switches a wedge 12b. Furthermore, the X-ray-irradiation control circuit 11 adjusts an irradiation range (a fan angle or a cone angle) of an X-ray by adjusting an opening degree of a collimator 12c. Note that in the present embodiment, it can be arranged such that more than one kind of wedge 12b is manually switched by an operator.

The X-ray generating device 12 is a device that generates an X-ray and irradiates the generated X-ray to the subject P, and includes the X-ray tube 12a, the wedge 12b, and the collimator 12c.

The X-ray tube 12a is a vacuum tube that irradiates an X-ray beam to the subject P by a high voltage supplied by the high-voltage generating unit not illustrated, and irradiates the X-ray beam onto the subject P with rotation of the rotating frame 15. The X-ray tube 12a generates an X-ray beam that radiates in a fan angle and a cone angle. For example, the X-ray tube 12a can emit an X-ray continuously all around the subject P for full reconstruction, or can emit an X-ray continuously in an irradiation range (180 degrees+fan angle) enabling half reconstruction for the half reconstruction, by the control of the X-ray-irradiation control circuit 11. Moreover, the X-ray tube 12a can emit an X-ray intermittently (pulsed X-ray) at a predetermined position (tube position) by the control of the X-ray-irradiation control circuit 11. Furthermore, the X-ray-irradiation control circuit 11 can modulate the intensity of an X-ray to be emitted from the X-ray tube 12a also. For example, the X-ray-irradiation control circuit 11 increases the intensity of an X-ray to be emitted from the X-ray tube 12a at a specific tube position, and decreases the intensity of an X-ray to be emitted from the X-ray tube 12a in a range other than the specific tube position.

The wedge 12b is an X-ray filter to adjust an amount of an X-ray that is emitted from the X-ray tube 12a. Specifically, the wedge 12b is a filter through which an X-ray irradiated from the X-ray tube 12a passes to be attenuated so that the X-ray to be irradiated to the subject P from the X-ray tube 12a has a predetermined distribution. For example, the wedge 12b is a filter that is obtained by processing aluminum to have a predetermined target angle and a predetermined thickness. The wedge 12b is called wedge filter, or bow-tie filter.

The collimator 12c is a slit to narrow an irradiation range of an X-ray, the amount of which has been adjusted by the wedge 12b, by the control of the X-ray-irradiation control circuit 11.

The base driving circuit 16 rotates the X-ray generating device 12 and the detector 13 on a circular orbit about the subject P in center, by driving the rotating frame 15 to be rotated.

The detector (X-ray detector) 13 is a two-dimensional array detector (surface detector) that detects an X-ray that has passed through the subject P, and has rows of detecting devices in which X-ray detectors for multiple channels are arranged aligned along a Z-axis direction. Specifically, the detector 13 in the first embodiment has X-ray detecting devices that are arranged in multiple rows of 320 rows along the Z-axis direction, and is capable of, for example, detecting an X-ray that has passed through the subject P in a wide range, such as a range including a lung and the heart of the subject P. The Z-axis direction indicates a direction of rotation center axis of the rotating frame 15 in a state in which the base 10 is not tilted.

The data collecting circuit 14 is a DAS, and collects projection data from detection data of an X-ray detected by the detector 13. For example, the data collecting circuit 14 performs amplification processing, analog-to-digital (A/D) conversion processing, sensitivity correction processing among channels, and the like on an X-ray-intensity distribution data that is detected by the detector 13, to generate projection data, and transmits the generated projection data to the console 30 described later. For example, when an X-ray is continuously emitted from the X-ray tube 12a while the rotating frame 15 is rotating, the data collecting circuit 14 collects a projection data group corresponding to all circumference (360 degrees). Moreover, the data collecting circuit 14 transmits the respective collected projection data associating with a tube position, to the console 30 described later. The tube position is information indicating a projection direction of the projection data. Note that the sensitivity correction processing among channels can be performed by preprocessing circuitry 34 described later.

The bed unit 20 is a device on which the subject P is placed, and as illustrated in FIG. 1, includes a bed driving device 21, and a top plate 22. The bed driving device 21 moves the top plate 22 in the Z-axis direction, and moves the subject P to the inside of the rotating frame 15. The top plate 22 is a plate on which the subject P is placed.

The base 10 rotates the rotating frame 15 while moving the top plate 22, for example, and performs helical scanning in which the subject P is scanned helically. Alternatively, the base 10 performs conventional scanning in which the subject P is scanned in a circular orbit by rotating the rotating frame 15 while the position of the subject P is fixed after the top plate 22 is moved. Alternatively, the base 10 performs step-and-shoot in which the conventional scanning is performed in more than one scanning area while changing the position of the top plate 22 at regular intervals.

The console 30 is a device that accepts an operation of the X-ray CT apparatus 1 by an operator, and that reconstructs X-ray-CT image data by using projection data collected by the base 10. The console 30 includes, as illustrated in FIG. 1, an input circuit 31, a display 32, the scan control circuitry 33, the preprocessing circuitry 34, storage 35, image reconstructing circuitry 36, and processing circuitry 37. The input circuit 31, the display 32, the scan control circuitry 33, the preprocessing circuitry 34, the storage 35, the image reconstructing circuitry 36, and the processing circuitry 37 are connected so as to be able to communicate with each other.

The input circuit 31 includes a mouse, a keyboard, a trackball, a switch, a button, a joystick, and the like that are used by an operator of the X-ray CT apparatus 1 to input various kinds of instructions and settings, and transfers information about instructions and settings received from an operator to the processing circuitry 37. For example, the input circuit 31 accepts an imaging condition of X-ray CT image data, a reconstruction condition at the time of reconstructing X-ray CT image data, an image processing condition for X-ray CT image data, and the like from the operator. Moreover, the input circuit 31 accepts an operation to select an examination for the subject P. Furthermore, the input circuit 31 accepts a specifying operation to specify a portion on an image.

The display 32 is a monitor that is referred to by an operator, and displays image data that is generated from X-ray CT image data to the operator, or displays a graphical user interface (GUI) to accept various kinds of instructions and settings and the like from the operator through the input circuit 31 under control of the processing circuitry 37. Moreover, the display 32 displays a plan screen of a scanning plan, a screen during scanning, and the like.

The scan control circuitry 33 controls collection processing of projection data in the base 10 by controlling operation of the X-ray-irradiation control circuit 11, the base driving circuit 16, the data collecting circuit 14, and the bed driving device 21, under control of the processing circuitry 37. Specifically, the scan control circuitry 33 controls collection processing of projection data in positioning imaging to collect a positioning image (scano-image), and actual imaging (main scanning) to collect an image to be used for diagnosis.

For example, the scan control circuitry 33 fixes the X-ray tube 12a at a position of 0 degree (position in a front direction relative to a subject), and acquires a two-dimensional scano-image by performing imaging continuously while moving the top plate 22 at a constant speed. Alternatively, the scan control circuitry 33 fixes the X-ray tube 12a at the position of 0 degree, and acquires a two-dimensional scano-image, while intermittently moving the top plate 22, by repeating intermittent imaging synchronized with movement of the top plate. The scan control circuitry 33 can capture a positioning image not only from the front direction relative to the subject P, but also from any direction (for example, from a side direction or the like).

Furthermore, the scan control circuitry 33 performs imaging of three-dimensional X-ray CT image data (volume data) by collecting projection data of all circumference of a subject. For example, the scan control circuitry 33 collects projection data corresponding to all circumference of the subject P by helical scanning or non-helical scanning. Moreover, the scan control circuitry 33 can acquire a three-dimensional scano-image by collecting projection data of all circumference with lower dose than that in actual scanning.

Furthermore, the scan control circuitry 33 can perform dynamic volume scanning (also referred to as “dynamic scanning”) to acquire plural pieces of volume data in temporal order by continuing imaging to acquire volume data for a predetermined period. For example, by collecting projection data of all circumference for a predetermined period while the subject P is doing an exercise of one joint, plural pieces of volume data that are reconstructed at a predetermined frame rate (volume rate) can be acquired. Temporal volume data that is acquired by dynamic scanning is called four-dimensional X-ray CT image data, or 4D CT image data.

The preprocessing circuitry 34 performs correction processing, such as logarithmic conversion processing, offset correction, sensitivity correction, and beam hardening correction, on the projection data generated by the data collecting circuit 14, to generate corrected projection data. Specifically, the preprocessing circuitry 34 generates corrected projection data for each of the projection data of the positioning image that is generated by the data collecting circuit 14 and projection data that is collected in the actual imaging, to store in the storage 35.

The storage 35 stores the projection data generated by the preprocessing circuitry 34. Specifically, the storage 35 stores the projection data of a positioning image, and the projection data for diagnosis collected in the actual imaging, generated by the preprocessing circuitry 34. Moreover, the storage 35 stores X-ray CT image data that is generated by the image reconstructing circuitry 36 described later.

Furthermore, the storage 35 stores, as necessary, a processing result by the processing circuitry 37 described later.

The image reconstructing circuitry 36 reconstructs X-ray-CT image data by using the projection data stored in the storage 35. Specifically, the image reconstructing circuitry 36 reconstructs X-ray CT image data from each of the projection data of the positioning image and the projection data of an image used for diagnosis. Various methods are available as a reconstruction method, and the back-projection processing is one, for example. Moreover, as the back-projection processing, for example, back projection processing by filtered back projection (FBP) can be applied. Alternatively, the image reconstructing circuitry 36 can reconstruct X-ray CT image data by using a method of successive approximation. Furthermore, the image reconstructing circuitry 36 generates image data by performing various kinds of image processing on X-ray CT image data. The image reconstructing circuitry 36 stores the reconstructed CT image data, and the image data that is generated by various kinds of image processing in the storage 35. The image reconstructing circuitry 36 is an example of an image reconstructing unit.

Moreover, the image reconstructing circuitry 36 reconstructs temporal three-dimensional medical-image data (4D CT image data) that is acquired by dynamic scanning. For example, the image reconstructing circuitry 36 reconstructs plural pieces of volume data in temporal order by reconstructing projection data of all circumference that is collected for a predetermined period continuously at a predetermined frame rate. Thus, for example, volume data (4D CT image data) of plural consecutive frames (phases) that shows movement of one joint. The image reconstructing circuitry 36 stores the reconstructed 4D CT image data in the storage 35.

The processing circuitry 37 performs overall control of the X-ray CT apparatus 1 by controlling operation of the base 10, the bed unit 20, and the console 30. Specifically, the processing circuitry 37 controls CT scanning performed in the base 10 by controlling the scan control circuitry 33. Moreover, the processing circuitry 37 controls the image reconstruction processing and the image generation processing in the console 30 by controlling the image reconstructing circuitry 36. Furthermore, the processing circuitry 37 controls to display various kinds of image data stored in the storage 35 on the display 32.

Moreover, the processing circuitry 37 performs an extraction function 371, a calculation function 372, a detection function 373, and an output control function 374 as illustrated in FIG. 1. Respective processing functions performed by the extraction function 371, the calculation function 372, the detection function 373, and the output control function 374 that are components of the processing circuitry 37 illustrated in FIG. 1 are recorded in the storage 35 in a form of programs that can be executed by a computer. The processing circuitry 37 is a processor that implements the functions corresponding to the respective programs by reading and executing the respective programs from the storage 35. In other words, the processing circuitry 37 that has read the respective programs is to have the respective functions indicated in the processing circuitry 37 in FIG. 1. The respective functions performed by the extraction function 371, the calculation function 372, the detection function 373, and the output control function 374 are described later.

Although the case in which the respective processing functions are implemented by a single unit of the processing circuitry 37 has been explained as an example in the present embodiment, the processing circuitry 37 can be configured by combining multiple independent processors such that the respective processors implement the respective processing functions by executing respective programs.

A term “processor” used in the above explanation signifies, for example, a circuit such as a central processing unit (CPU), a graphical processing unit (GPU), an application specific integrated circuit (ASIC), a programmable logic device (for example, a simple programmable logic device (SPLD), and a complex programmable logic device (CPLD)), and a field programmable gate array (FPGA). The processor reads and executes a program stored in the storage 35, and thereby implements a function. Instead of storing a program in the storage 35, the program can be directly installed in a circuit of the processor. In this case, the processor reads and executes the program installed in the circuit of the processor to implement the function. The respective processors in the present embodiment is not limited to be structured as an independent circuit per processor, but can be structured by combining multiple independent processors to form a single processor to implement the functions. Furthermore, more than one component in each drawing can be integrated to a single processor to implement the functions.

As above, the configuration of the X-ray CT apparatus 1 according to the first embodiment has been explained. With the configuration, the X-ray CT apparatus 1 according to the first embodiment performs the following respective processing functions to detect a region of a lung having an abnormality in the respiratory activity accurately.

The extraction function 371 extracts plural regions corresponding to at least one of a lobe and a subsegment forming a lung from three-dimensional medical-image data that is obtained by imaging temporally. For example, the extraction function 371 reads 4D CT image data stored in the storage 35 from the storage 35. The extraction function 371 then extracts a region corresponding to an entire lung from the read 4D CT image data based on a CT value. The extraction function 371 extracts plural regions corresponding to a subsegment from among regions of the entire lung by segmentation. The extraction function 371 is an example of an extracting unit.

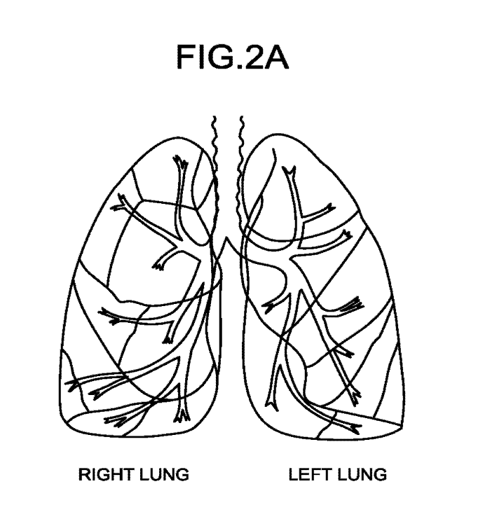

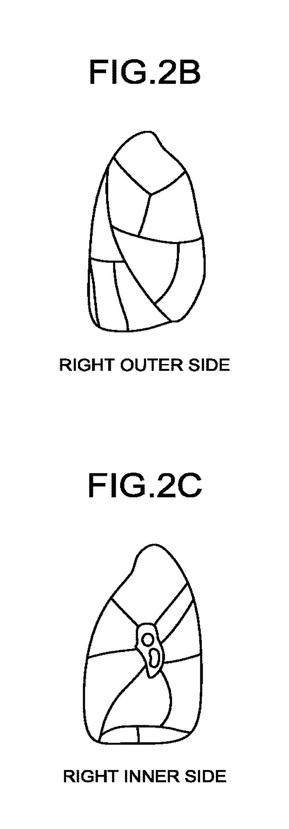

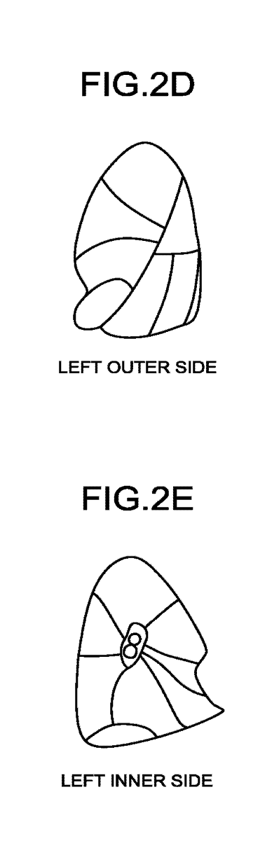

FIG. 2A to FIG. 2E are diagrams for explaining processing of the extraction function 371 according to the first embodiment. FIG. 2A illustrates a schematic diagram of right and left lungs that are viewed from the front. FIG. 2B illustrates a schematic diagram of a right outer side (outer side of the right lung). Moreover, FIG. 2C illustrates a schematic diagram of a right inner side (inner side of the right lung). FIG. 2D illustrates a schematic diagram of a left outer side (outer side of the left lung). Furthermore, FIG. 2E illustrates a schematic diagram of a left inner side (inner side of the left lung).

As illustrated in FIG. 2A to FIG. 2E, the extraction function 371 extracts, for example, plural regions respectively corresponding to plural subsegments from an entire lung region. The subsegment is a region that forms a lobe of a lung. As a specific example, the extraction function 371 extracts plural regions respectively corresponding to subsegments of a subject by transforming a template image that indicates positions of subsegments in a lung into a lung shape of the subject. With the template image, three-dimensional positional relationships of anatomical characteristics of a lung have been associated in advance.

The processing of the extraction function 371 described above is only one example, and is not limited to the above example. For example, although the case in which the extraction function 371 extracts regions corresponding to subsegments of a lung has been explained in the above example, a region corresponding to a lobe can be extracted. Moreover, although the case of using a template image has been explained in the above explanation, it is not limited thereto. For example, by analyzing movement of a lung in 4D CT image data, they can be extracted assuming a portion making different movement as a boundary of lobes and subsegments. Moreover, as a method of extracting a lobe and a subsegment, any conventional technique can be applied.

The calculation function 372 calculates a physical index (parameter) relating to respiratory activity for each of the extracted regions. For example, the calculation function 372 calculates a volume of each of the regions extracted by the extraction function 371. As a specific example, the calculation function 372 calculates a volume of each of the regions for volume data of each time phase that is included in 4D CT image data. Thus, the calculation function 372 calculates a volume at each time phase of each of the plural regions. The calculation function 372 is an example of a calculating unit.

Note that the processing of the calculation function 372 described above is only one example, and it is not limited to the above example. For example, although the case in which the calculation function 372 calculates a volume as the physical index has been explained in the above example, it is not limited thereto. For example, a surface area, a specific surface area (value obtained by dividing surface area by volume), or a CT value can be calculated. A CT value of each region is a mean value of CT values of all pixels that are included in each region, and is used as an index that expresses the respiratory activity because the CT value varies according to an amount of air included in each region. That is, the calculation function 372 can calculate at least one of a volume, a surface area, a specific surface area, and a CT value.

The detection function 373 detects an abnormal region in which the respiratory activity is abnormal, out of the plural regions based on a temporal change of the physical index. For example, the detection function 373 detects an abnormal region by comparing a temporal change of the physical index of plural regions. The detection function 373 is an example of a detecting unit. In other words, the detection function 373 detects a region that is abnormal in the respiratory activity out of the plural regions by comparing a temporal change of the physical index of the respective regions with each other. A temporal change also referred to as “chronological change”, “transition”, “time course” and so on.

For example, the detection function 373 generates a curve that indicates a temporal change of a parameter of each region, by drawing a fitting curve of values of the parameter at each time phase of the regions. The detection function 373 then detects a maximum point and a minimum point from each of the generated curves. The maximum point corresponds to “maximum inhalation”, and the minimum point corresponds to “maximum exhalation”. The detection function 373 detects a region in which the tendency of the curve of the temporal change of a parameter is different among the plural regions, as an abnormal region.

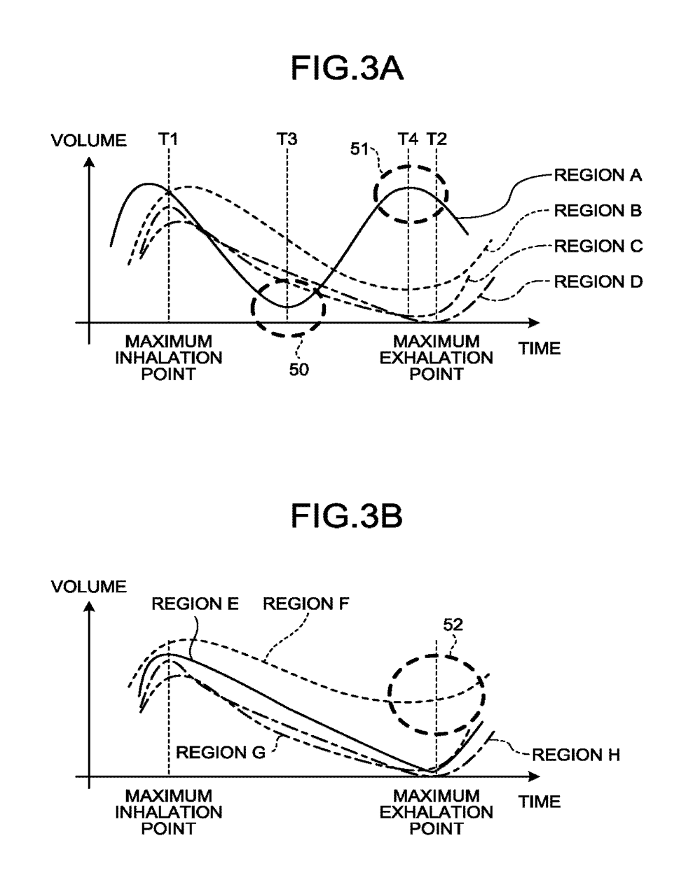

FIG. 3A and FIG. 3B are diagrams for explaining processing of the detection function 373 according to the first embodiment. FIG. 3A illustrates a graph (curve) that expresses a temporal change of the volumes of region A to region D. Moreover, FIG. 3B illustrates a graph that expresses a temporal change of the volumes of region E to region H. In FIG. 3A and FIG. 3B, a vertical axis is for volume of each region, and a horizontal axis is for time. Note that regions A to H correspond to respective subsegments. Furthermore, in FIG. 3A, the maximum inhalation point of regions B to D is “T1”, and the maximum exhalation point thereof is “T2”. Moreover, the maximum inhalation point of region A is “T3”, and the maximum exhalation point thereof is “T4”.

As illustrated in FIG. 3A, the detection function 373 detects an abnormal region based on a time lag in the maximum inhalation point and the maximum exhalation point. For example, the detection function 373 calculates a difference between the maximum inhalation points of the respective regions, and compares the differences with each other, thereby detecting a time lag in the maximum inhalation point and the maximum exhalation point. In FIG. 3A, the maximum exhalation point T3 (minimum point at 50) of region A is different from the respective maximum exhalation points T2 of the other regions B to D. Specifically, even if the maximum inhalation point of region A is shifted to substantially match with the maximum inhalation point T1 of respective regions B to D, the following maximum exhalation point is different from others. Similarly, the maximum inhalation point T4 (maximum point at 51) of region A is different from the respective maximum inhalation points of the other regions B to D. In this case, the detection function 373 detects region A as an abnormal region. In other words, the detection function 373 detects a region as an abnormal region when a time lag in the maximum exhalation point (or the maximum inhalation point) thereof is equal to or larger than a threshold.

As illustrated in FIG. 3B, the detection function 373 detects an abnormal region based on a difference between volumes at the maximum inhalation and the maximum exhalation (peak to peak: P-P). For example, the detection function calculates P-P of respective regions, and compares them with each other, thereby detecting an abnormal region. FIG. 3B shows that P-P of region F is small compared to P-P of the other regions E, G, and H, and that air is not completely exhaled therein (minimum point at 52). In this case, the detection function 373 detects region F as an abnormal region.

As described, the detection function 373 compares tendencies of curves that indicate a temporal change of a parameter of plural regions. Thus, the detection function 373 detects a region that differs in tendency from other regions, as an abnormal region.

The processing of the detection function 373 described above is only one example, and it is not limited to the above example. For example, the vertical axis can be expressed by a ratio (%) the maximum volume of each region. Moreover, although the case in which the detection function 373 detects an abnormal region based on a time lag of the maximum inhalation point and the maximum exhalation point in the above example, embodiments are not limited thereto. For example, the detection function 373 can detect an abnormal region by using a time lag of either one out of the maximum inhalation and the maximum exhalation. Furthermore, for example, the detection function 373 can detect a region that cannot be approximated to a sine curve, or a region with non-periodic curve, as an abnormal region.

Moreover, although the case of detecting a region in which the tendency of a curve is different as an abnormal region has been explained in the above example, embodiments are not limited thereto. Other detecting methods are described later as modifications.

The output control function 374 outputs information that indicates about an abnormal region. For example, the output control function 374 causes an abnormal region to be displayed in an emphasized manner in three-dimensional medical-image data of a time phase at which the abnormal region is detected. The output control function 374 is an example of an output control unit.

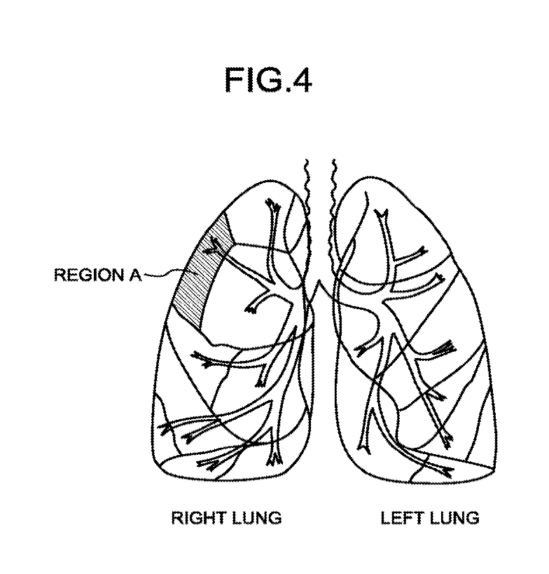

FIG. 4 is a diagram for explaining processing of the output control function 374 according to the first embodiment. FIG. 4 illustrates an image of a lung of a subject that is displayed on the display 32. FIG. 4 shows a case in which “region A” has been detected as an abnormal region.

As illustrated in FIG. 4, for example, the output control function 374 causes the display 32 to display an image of a lung based on three-dimensional medical-image data. This image is a volume rendered image (or multi-planner reconstruction (MPR) image, or the like) based on three-dimensional medical-image data at a time phase (T3 or T4) at which region A has been detected. In other words, the output control function 374 displays an image for display based on three-dimensional medical-image data of a time phase at which an abnormal region is detected. The output control function 374 then displays a portion in the image corresponding to the abnormal region that has been detected by the detection function 373 in an emphasized manner (by displaying in a different color from other regions, or the like).

As described, the output control function 374 causes the display 32 to display information about an abnormal region. FIG. 4 is only one example, and it is not limited to the illustrated example. For example, the output control function 374 can display an abnormal region in an image of any time phase, not necessarily limited to an image that is based on three-dimensional medical-image data of a time phase at which the abnormal region is detected.

Moreover, although the case in which an image of a lung is displayed as a still image has been explained in FIG. 4, embodiments are not limited thereto. For example, the output control function 374 can display an image of a lung as a moving image. In a moving image, the output control function 374 can display an abnormal region in such a manner that the abnormal region is emphasized as a time phase at which the abnormal region is detected approaches, and the emphasized display is canceled (not emphasized) as the time phase at which the abnormal region is detected goes away.

Furthermore, although the case in which information about an abnormal region is displayed on the display 32 has been explained in FIG. 4, embodiments are not limited thereto. For example, the output control function 374 can output it as text data, such as “region A”, or can output as voice by a text reading function. Specifically, the output control function 374 can display (or output a voice) information (time) indicating a time phase (T3) at which an abnormal region is detected, or information (for example, difference between T2 and T3) indicating a magnitude of time lag that is detected by the detection function 373. Moreover, a destination to which the information about an abnormal region is output is not limited to the display 32 or a voice output device, but can be an arbitrary storage medium or another device (application to generate a report, and the like).

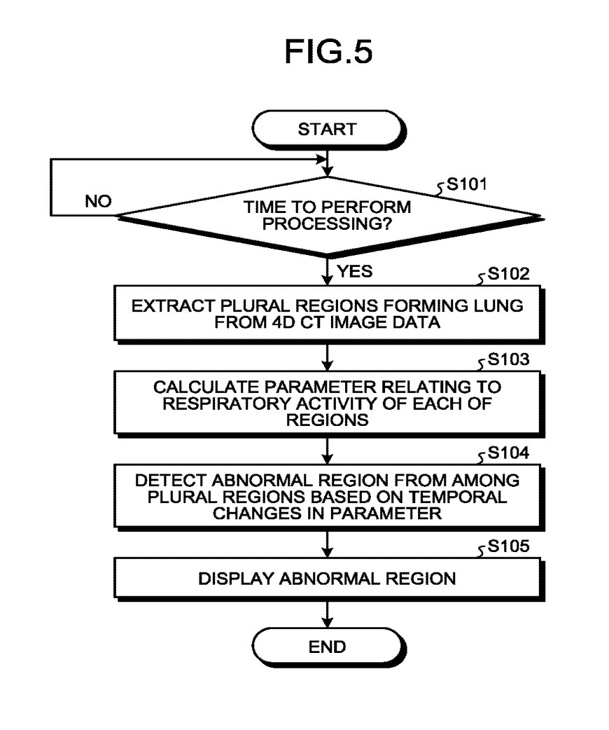

FIG. 5 is a flowchart of a procedure of processing performed by the X-ray CT apparatus 1 according to the first embodiment. The procedure illustrated in FIG. 5 is started, for example, when an instruction to start the processing to detect an abnormal region is input by an operator.

As illustrated in FIG. 5, at step S101, the processing circuitry 37 determines whether it is time for the processing. For example, the processing circuitry 37 determines that it is time for the processing when an instruction to start the processing to detect an abnormal region is input by an operator, and starts processing at step S102 and later. When a negative determination is made at step S101, the processing circuitry 37 does not start the processing at step S102 and later, and goes into a stand-by state.

When a positive determination is made at step S101, the extraction function 371 extracts plural regions that form a lung from 4D CT image data at step S102. For example, the extraction function 371 extracts a region corresponding to the entire lung from the 4D CT image data based on a CT value. The extraction function 371 then extracts plural regions corresponding to subsegments from the region corresponding to the entire lung by segmentation.

At step S103, the calculation function 372 calculates a parameter (physical index) relating to respiratory activity of each of the plural regions. For example, the calculation function 372 calculates a volume of each of the plural regions for volume data of respective time phases included in the 4D CT image data. Thus, the calculation function 372 calculates a volume at each time phase for each of the plural regions.

At step S104, the detection function 373 detects an abnormal region from among the plural regions based on a temporal change of a parameter. For example, the detection function 373 compares tendencies of curves that indicate a temporal change of the volumes of the respective regions with each other. The detection function 373 then detects an abnormal region based on time lags of the maximum inhalation and the maximum exhalation.

At step S105, the output control function 374 displays the abnormal region. For example, the output control function 374 displays the abnormal region in an emphasized manner in three-dimensional medical-image data of a time phase at which the abnormal region is detected.

As described, the X-ray CT apparatus 1 accepts an instruction of an operator to start the processing to detect an abnormal region, and performs the processing at respective steps S102 to S105, to display the abnormal region. What is described in FIG. 5 is only one example, and it is not limited thereto.

As described above, in the X-ray CT apparatus 1 according to the first embodiment, the extraction function 371 extracts plural regions corresponding to at least one of the lobe and the subsegment forming a lung from three-dimensional medical-image data that is obtained by imaging temporally. The calculation function 372 calculates a physical index (parameter) relating to respiratory activity for each of the extracted plural regions. The detection function 373 detects an abnormal region in which the respiratory activity is abnormal out of the plural regions based on a temporal change of the physical index. That is, the detection function 373 detects an abnormal region by comparing a temporal change of the physical index of the respective regions. The output control function 374 outputs information indicating about the abnormal region. According to this, the X-ray CT apparatus 1 can detect a region of a lung having an abnormality in the respiratory activity accurately.

For example, the X-ray CT apparatus 1 evaluates a state of a lung of the subject P not as a whole, but in a unit of a lobe or a subsegment. Thus, the X-ray CT apparatus 1 can detect not only whether the lung of the subject P has an abnormality, but also which part (region) of the lung has an abnormality, and present it to an operator.

### First Modification of First Embodiment

The processing of the detection function 373 is not limited to the embodiment described above, but can be implemented by other embodiments. For example, the detection function 373 can detect a region having an evaluation value (index) based on a time lag of the maximum inhalation and the maximum exhalation in a temporal change of a parameter, as an abnormal region.

For example, the detection function 373 calculates an evaluation value F [%] by using Equation (1) below. In Equation (1), Vi corresponds to a volume at the time of maximum inhalation. Moreover, Ve corresponds to a volume at the time of maximum exhalation.

$$F ( \% ) = \frac{{V}_{i} - {V}_{e}}{{V}_{i}} \times 100 \qquad ( 1 )$$

That is, the detection function 373 acquires the evaluation value F [%] by dividing a difference between a value at the maximum inhalation and a value at the maximum exhalation by the volume at the maximum inhalation to express in percentage, for each of plural regions. Subsequently, the detection function 373 compares the calculated evaluation values F [%] of the respective regions with each other. Based on a result of comparison, the detection function 373 detects a region, the evaluation value F [%] of which is low compared to that of the other regions as an abnormal region.

As described, the detection function 373 detects a region in which an evaluation value based on a difference in a parameter between the maximum inhalation point and the maximum exhalation point in a temporal change thereof is lower than that of the other regions as an abnormal region. The evaluation value F [%] can be calculated by using a surface area, a specific surface area, or a CT value, not limited to a volume.

In the first modification, an abnormal region can be detected as long as volume data at the time of maximum inhalation and the maximum exhalation is available. In other words, the detection function 373 can detect an abnormal region without using 4D CT image data, by using volume data that is imaged while the subject P holds breath at the time of the maximum inhalation (or the maximum exhalation).

### Second Modification of First Embodiment

Moreover, for example, the detection function 373 can detect a region in which a differential coefficient of a period including the maximum exhalation point in a temporal change of a parameter is smaller than that of the other regions, as an abnormal region.

For example, as one of important signs of a pulmonary disease, whether breath is fully exhaled is considered. It is thought that a curve around the maximum exhalation is gentle when breath is not fully exhaled.

Therefore, the detection function 373 identifies the minimum point (downward inflection point) from a curve expressing a temporal change of a parameter, for each of plural regions. The detection function 373 then calculates a differential coefficient of a curve of a predetermined period that includes a time phase of the identified minimum point. The detection function 373 compares the calculated differential coefficients of the respective regions with each other. Based on a result of comparison, the detection function 373 detects a region, the differential coefficient of which is smaller than that of the other regions as an abnormal region.

As described, the detection function 373 detects a region in which a differential coefficient of a period including the maximum exhalation point in a temporal change of a parameter is smaller than that of the other regions as an abnormal region. The period for calculating a differential coefficient can be arbitrarily set. Moreover, the detection function 373 can calculate a differential coefficient of a predetermined period including the maximum inhalation point.

### Third Modification of First Embodiment

Furthermore, for example, the detection function 373 can detect an abnormal region by comparing regions corresponding to each other in left and right lungs.

For example, there is a case that the subject P has a subjective symptom, as for in which one of the left and right lungs there is an abnormality. In such a case, by comparing regions corresponding to each other in the left and right lungs, detection of an abnormal region is enabled.

For example, the detection function 373 compares a temporal change of a parameter in each region with a temporal change of a parameter in a region corresponding to the region. Note that lobes and subsegments forming left and right lungs are not necessarily positioned symmetrically. Therefore, the detection function 373 compares a region of subject with a region at the most approximate position to a position symmetrical to the region of subject.

As one example, when the subject P feels that the left lung has an abnormality and the right lung is normal, the detection function 373 compares each region of the left lung with a region at the most approximate position to a position symmetrical to that in the right lung. The detection function 373 then detects a region in the left lung that significantly differs in tendency of a temporal change (curve) of a parameter from the region in the right lung, as an abnormal region.

As described, the detection function 373 detects a region that is significantly different when compared with a normal one out of left and right lungs, as an abnormal region. Although the case of comparing subsegments with each other has been explained in the above explanation, it is not limited thereto, and the comparison can be performed in a unit of lobes.

### Fourth Modification of First Embodiment

Moreover, for example, the detection function 373 can detect an abnormal region by comparing each region with a reference region to be a reference out of plural regions.

For example, the detection function 373 sets a region that has a standard value for the CT value as a reference region (normal region) out of plural regions. The detection function 373 then calculates a relative value by dividing a parameter of each of the plural regions by a parameter of the reference region. When respective regions are normal, the relative values of respective regions take substantially the same value. Therefore, the detection function 373 compares the relative values of the respective regions with each other, and detects a region, the relative value of which is different compared with those of the other regions, as an abnormal region. The CT value of a normal subsegment is set in advance.

As described, the detection function 373 can detect an abnormal region by comparing the reference region to be a reference out of plural regions with each region. Although the case in which a region, the CT value of which is a standard value is used as the reference region has been explained in the above explanation, it is not limited thereto. For example, the detection function 373 can set a region in which the change between volumes at the maximum inhalation and the maximum exhalation is the largest, or a region that is specified by an operator as the reference value. That is, the detection function 373 sets a region regarded as a normal subsegment, as the reference region.

### Fifth Modification of First Embodiment

Furthermore, for example, the detection function 373 can detect a region in which a value of a predetermined time phase in a curve of a temporal change of a parameter is smaller than a value of the predetermined time phase when the parameter changes according to a sine curve, as an abnormal region.

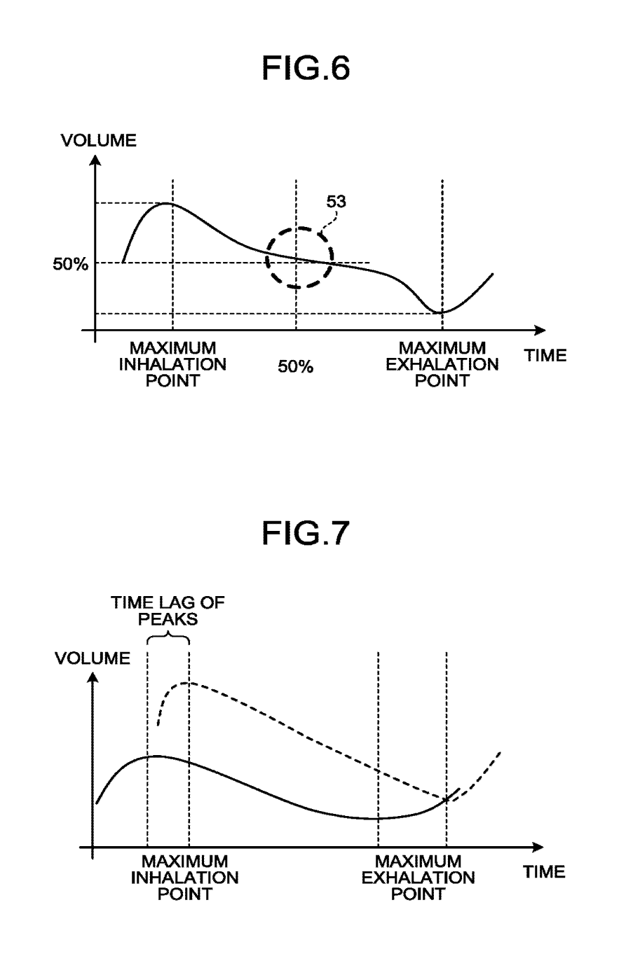

FIG. 6 is a diagram for explaining processing of the detection function 373 according to a modification of the first embodiment. FIG. 6 illustrates a graph that shows a temporal change of the volume in one region. In FIG. 6, a vertical axis is for volume of each region, and a horizontal axis is for time.

As illustrated in FIG. 6, for example, the detection function 373 calculates a volume of one-time phase as a threshold, assuming that a curve of a temporal change of the volume of one region is to be a sine curve. In the example of FIG. 6, the detection function 373 calculates a value at a midpoint (50%) between the maximum inhalation point and the maximum exhalation point as the threshold. The detection function 373 then detects an abnormal region based on determination whether the curve of a temporal change of the volume of one region reaches the threshold. In the example of FIG. 6, the curve of one region does not reach 50% at a midpoint 53. Therefore, the detection function 373 detects this region as an abnormal region.

As described, the detection function 373 detects a region in which a value of a predetermined time phase of a curve of a temporal change of a parameter does not reach a value of a predetermined time phase when the parameter changes according to a sine curve, as an abnormal region. FIG. 6 illustrates only one example, and it is not limited to the above explanation. For example, although the case in which a value of the time phase at the midpoint between the maximum inhalation point and the maximum exhalation point is used for the threshold has been explained, it is not limited thereto, and a value at any time phase can be set thereto. Moreover, although the case of setting a value of one time phase as the threshold has been explained, it is not limited thereto, and values of more than one time phase can be set as the threshold.

### Second Embodiment

While the case in which the X-ray CT apparatus 1 detects a region corresponding to an abnormal lobe or subsegment has been explained in the first embodiment, embodiments are not limited thereto. For example, the X-ray CT apparatus 1 can perform processing of detecting an abnormal region also in a bronchus to supply air to lobes and subsegments.

The X-ray CT apparatus 1 according to a second embodiment has the same configuration as the X-ray CT apparatus 1 illustrated in FIG. 1, but differs a part of the processing of the processing circuitry 37. Therefore, in the second embodiment, points that differ from the first embodiment is mainly explained, and explanation about portions having functions similar to those of the components explained in the first embodiments is omitted.

For example, the extraction function 371 further extracts plural bronchus regions corresponding to a bronchus to supply air to each of the plural regions. Normally, a human bronchus is branched main bronchi (left main bronchus, right main bronchus) to supply air to left and right lungs, and is further branched to lobar bronchi to supply air to lobes and subsegmental bronchi (end portions) to supply air to subsegments. For example, the extraction function 371 extracts a region that corresponds to subsegmental bronchi to supply air to a subsegment region, as a bronchus region. As for the extraction method, any conventional technique, such as a method of using a template image, can be applied.

A range to be extracted by the extraction function 371 as a bronchus region is not limited to a subsegmental bronchus. For example, the extraction function 371 can extract a range that includes a lobar bronchus, and left and right main bronchi, in addition to a subsegmental bronchus, as a bronchus region. However, it is preferable that a region including at least a subsegmental bronchus be extracted, to obtain correspondence with a subsegment.

For example, the calculation function 372 calculates a physical index for each of the extracted bronchus regions. As a specific example, the calculation function 372 calculates a surface area, a specific surface area, or a CT value as the physical index also for bronchus regions, similarly to regions of subsegment. Moreover, the calculation function 372 can calculate a cross-sectional area of each of the bronchus regions as the physical index of bronchus regions. For example, the cross-sectional area is an area of a cross-section perpendicular to a longitudinal axis of a bronchus region.

For example, the detection function 373 detects an abnormal region by comparing sets of a region and a bronchus region in correspondence. The region and the bronchus region in correspondence signifies a region of a subsegment and a bronchus region that includes a subsegmental bronchus to supply air to the subsegment.

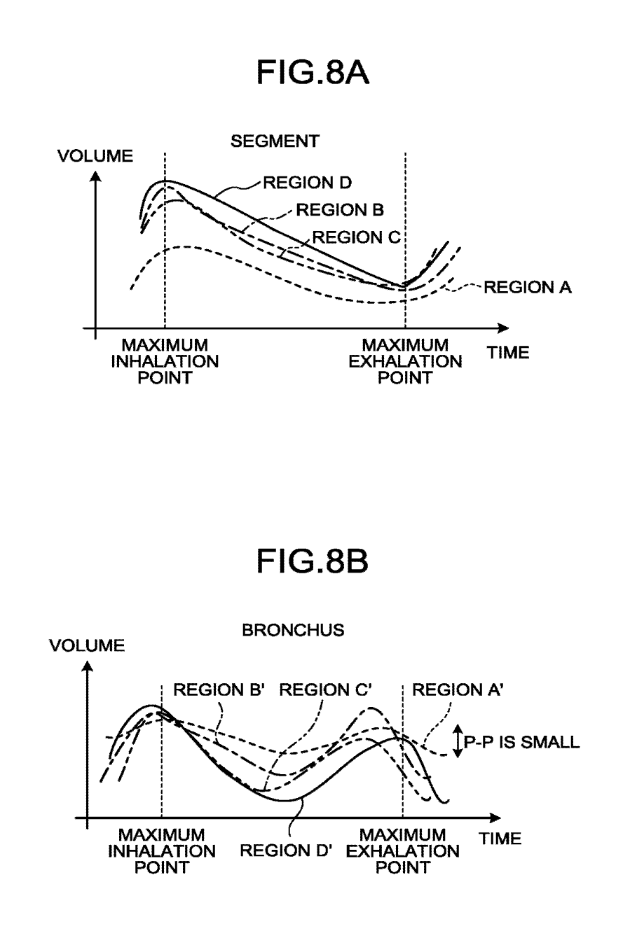

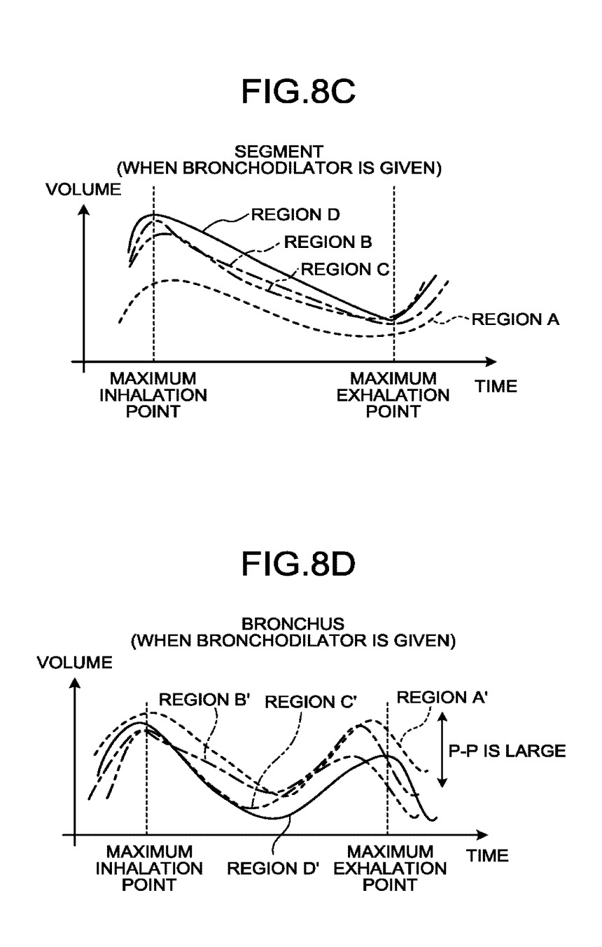

FIG. 7 and FIG. 8A to FIG. 8D are diagrams for explaining the processing of the detection function according to the second embodiment. In FIG. 7 and FIG. BA to FIG. 8D, a vertical axis is for volume of each region, and a horizontal axis is for time.

With the example illustrated in FIG. 7, the case in which the detection function 373 detects an abnormal region by using a time lag between peaks of a region and a bronchus region in correspondence is explained. There is an anatomical relationship between a subsegment and a subsegmental bronchus in correspondence that the subsegmental bronchus expands first, and then the subsegment expands. The time lag of these expansions is considered to be approximately uniform among the respective subsegments. Therefore, the detection function 373 calculates a time lag of peaks in curves of temporal changes of a parameter for each pair (set) of a subsegment and a subsegmental bronchus in correspondence. In the example illustrated in FIG. 7, the detection function 373 calculates a difference between the maximum inhalation point in a curve (solid line) of a bronchus region and a maximum inhalation point of a curve (broken line) of a region of a subsegment. Subsequently, the detection function 373 compares time lags of peaks of respective pairs, and a region of a subsegment having a large time lag of peaks compared with the other pairs as an abnormal region.

With the example illustrated in FIG. 8A to FIG. 8D, a case of detecting an abnormal region by using bronchodilator is explained. FIG. 8A illustrates a temporal change (curve) in volume of a region of a subsegment when the bronchodilator is not given. Furthermore, FIG. 8B illustrates a temporal change (curve) in volume of a bronchus region when the bronchodilator is not given. Furthermore, FIG. 8C illustrates a temporal change (curve) in volume of the region of a subsegment when the bronchodilator is given. Moreover, FIG. 8D illustrates a temporal change (curve) in volume of the bronchus region when the bronchodilator is given. Regions A to D signify regions of a subsegment. Regions A′ to D′ signify bronchus regions to supply air to regions A to D, respectively.

As illustrated in FIG. 8A and FIG. 8C, no significant change is found in the temporal change of the volume of the subsegment between when the bronchodilator is given and not given. On the other hand, as illustrated in FIG. 8B and FIG. 8D, while P-P of region A′ when the bronchodilator is not given is small (FIG. 8B), P-P of region A′ when the bronchodilator is given is large (FIG. 8D). This is a sign of a COPD of an emphysema type.

Therefore, the detection function 373 calculates P-P of a region of a subsegment and a bronchus region when the bronchodilator is given and not given, and compares the calculated P-P with each other. When P-P of the region of a subsegment is not changed and P-P of the bronchus region is increased by giving the bronchodilator, the detection function 373 detects the region of a subsegment as an abnormal region.

As described, the detection function 373 detects an abnormal region by comparing sets of a region of a subsegment and a bronchus region in correspondence. FIG. 7 and FIG. 8A to FIG. 8D are only one example, and it is not limited to the explanation above. For example, when a bronchus is considered to be abnormal, the detection function 373 can detect an abnormal bronchus region that shows abnormal respiratory activities.

For example, the output control function 374 displays an image of a region of a subsegment and an image of a bronchus region to supply air to the region of a subsegment at the same time.

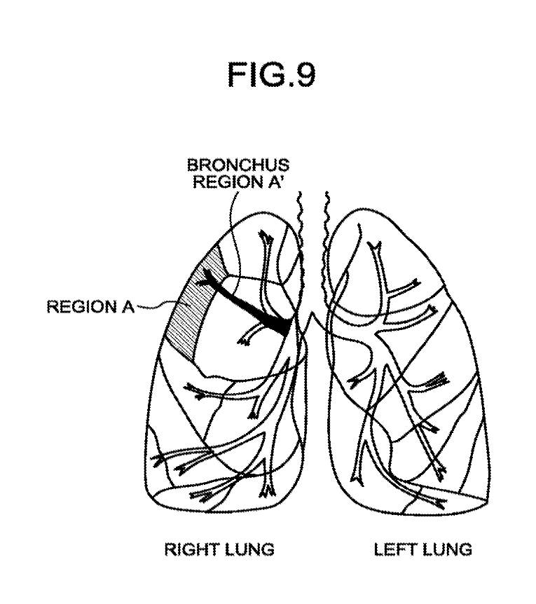

FIG. 9 is a diagram for explaining processing of the output control function 374 according to the second embodiment. FIG. 9 illustrates an image of a lung of a subject that is displayed on the display 32. FIG. 9 illustrates a case in which “region A” is detected as an abnormal region.

As illustrated in FIG. 9, for example, the output control function 374 displays an image of a lung based on three-dimensional medical-image data on the display 32. The output control function 374 highlights (by displaying in a different color from other regions, or the like) positions of region A that is detected by the detection function 373 and region A′ (bronchus region) corresponding to a bronchus to supply air to region A in the image.

As described, the output control function 374 displays information indicating about an abnormal region on the display 32. FIG. 9 illustrates only one example, and it is not limited to the illustrated example. For example, the output control function 374 can display a graph of a temporal change of a region of a subsegment and a graph of a temporal change of a bronchus region to supply air to the region of a subsegment at the same time.

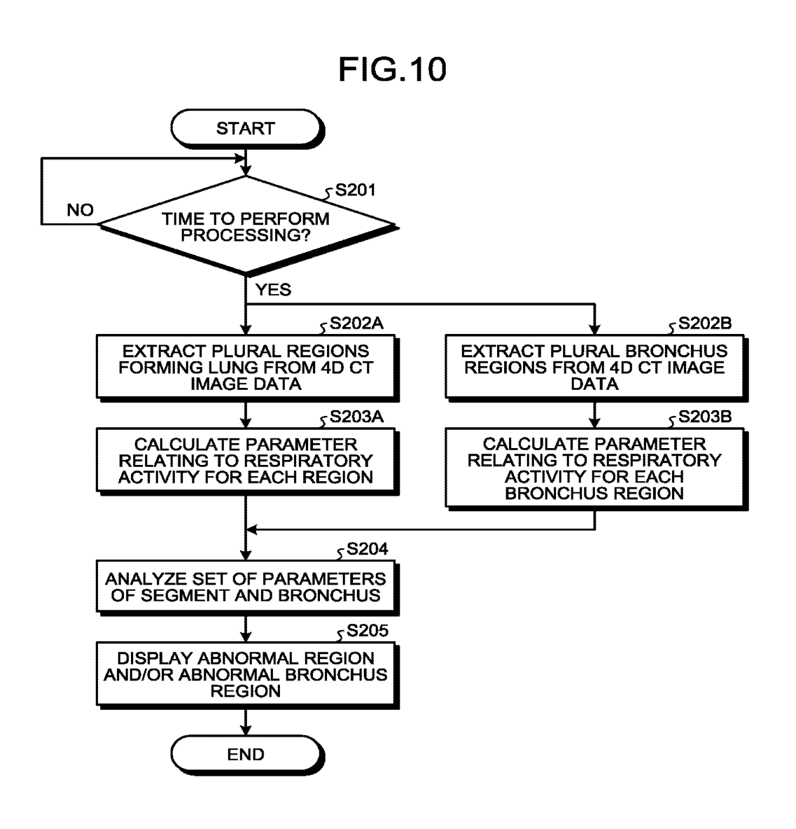

FIG. 10 is a flowchart of a procedure of processing performed by the X-ray CT apparatus 1 according to the second embodiment. Out of the procedure illustrated in FIG. 10, processing of step S201, step S202A, and step 203A is the same as the processing of step S101, step S102, and step S103 in FIG. 5, respectively, and therefore, explanation thereof is omitted.

As illustrated in FIG. 10, when a positive determination is made at step S201, the extraction function 371 extracts plural bronchus regions from 4D CT image data at step S202B. For example, the extraction function 371 extracts plural bronchus regions corresponding to a bronchus by using a template image.

At step S203B, the calculation function 372 calculates a parameter (physical index) relating to the respiratory activity for each bronchus. For example, the calculation function 372 calculates a volume of each of the bronchus regions for volume data of each time phase included in the 4D CT image data.

At step S204, the detection function 373 analyzes the parameter by combining parameters of a subsegment and a bronchus. For example, the detection function 373 detects an abnormal region by comparing sets of a region and a bronchus region in correspondence.

At step S205, the output control function 374 displays an abnormal region and/or an abnormal bronchus region. For example, the output control function 374 highlights the abnormal region and a bronchus region in correspondence with the abnormal region in three-dimensional medical-image data of a time phase at which the abnormal region is detected.

As described, the X-ray CT apparatus 1 accepts an instruction to start the processing of detecting an abnormal region from an operator, and performs the respective processing of steps S202 to S205, to display an abnormal region. The procedure in FIG. 10 is only one example, and it is not limited thereto. For example, although the case in which the processing at steps S202A, S203A, and the processing at steps S202B, S203B are performed by parallel processing has been explained in FIG. 10, embodiments are not limited thereto. For example, the processing at steps S202A, 203A and the processing at steps S202B, S203B are not necessarily performed by parallel processing. That is, out of the processing at steps S202A, S203A, and the processing at steps S202B, S203B, either one can be processed prior to the other.

As described above, the X-ray CT apparatus 1 according to the second embodiment performs the processing of detecting a region having an abnormality also for a bronchus to supply air to lobes and subsegments. Thus, for example, the X-ray CT apparatus 1 can analyze a lung of the subject P more precisely.

What has been explained in the first embodiment is also applicable to the second embodiment except the point that processing relating to a bronchus is performed.

### Other Embodiments

In addition to the embodiments described above, it can be implemented by various other embodiments.

Display of Graph when Respiratory Activity is Normal

In addition to the embodiments described above, it is possible to generate and display a graph when the respiratory activity in an abnormal region is normal.

For example, the output control function 374 displays a normal graph that shows a temporal change of a physical index when the respiratory activity is normal in an abnormal region. Specifically, the output control function 374 generates a normal graph by changing a waveform of a normal graph template based on a volume of the abnormal region, and a time phase of a region other than the abnormal region out of plural regions. The output control function 374 then displays the generated normal graph.

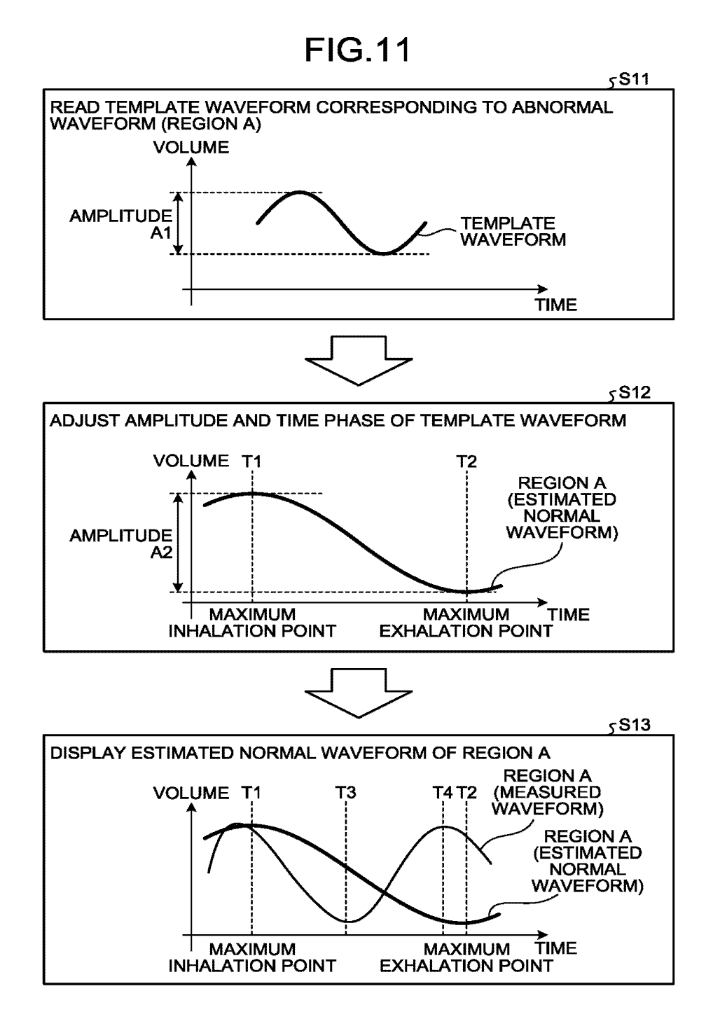

FIG. 11 is a diagram for explaining processing performed by an X-ray CT apparatus according to another embodiment. FIG. 11 illustrates a case in which processing of reading a template waveform corresponding to an abnormal region (region A) (S11), processing of adjusting an amplitude and a time phase of the template waveform (S12), and processing of displaying an estimated normal waveform of region A (S13) are sequentially performed. In FIG. 11, processing of displaying a normal graph of region A when region A is detected as an abnormal region out of regions A to D illustrated in FIG. 3A is explained.

As illustrated in FIG. 11, at S11, the processing of reading a template waveform corresponding to an abnormal waveform (region A) is performed. In the storage 35, the template waveform is stored in advance. The template waveform is, for example, a representative graph (curve) that expresses a temporal change of the volume of respective regions (lobes or subsegments) that form a lung. Subsequently, the output control function 374 reads a template waveform of a region corresponding to region A from the storage 35 when region A is detected as an abnormal region. The representative graph that expresses a temporal change of the volume of each region is determined by statistical processing of a temporal change of the volume of each region of multiple healthy people, but it is not limited thereto. For example, a sine waveform can be used simply as the template waveform.

At S12, the processing of adjusting an amplitude and a time phase of the template waveform is performed. For example, the output control function 374 adjusts amplitude A1 of the template waveform to amplitude A2 based on the volume of region A of the subject P. Specifically, the output control function 374 adjusts amplitude A1 to amplitude A2 based on comparison between a volume of region A of representative healthy people and the volume of region A of the subject P. More specifically, the output control function 374 acquires amplitude A2 by “A2=A1×V1/V0” when an average volume of region A of the representative healthy people is “V0” and an average volume of region A of the subject P is “V1”.

Moreover, the output control function 374 adjusts the time phase of the template waveform based on a time phase of graphs of normal regions B to D. Specifically, the output control function 374 deforms the template waveform in a temporal direction such that a time phase of the maximum inhalation point in the template waveform matches with “T1”, and a time phase of the maximum exhalation point in the template waveform matches with “T2”.

As described, the output control function 374 generates a graph of an estimated normal waveform of region A by adjusting the amplitude and the time phase of the template waveform. The graph of an estimated normal waveform is one example of a normal graph.

At S13, the processing of displaying the estimated normal waveform of region A is performed. For example, the output control function 374 displays the generated graph of the estimated normal waveform together with a measured waveform of region A on the display 32. The measured waveform of region A is the graph of region A in FIG. 3A.

The output control function 374 is not necessarily required to display it with the measured waveform of region A. For example, the output control function 374 can display only the graph of the estimated normal waveform of region A, or can display along with a measured waveform of the other regions B to D.

### Medical Image-Processing Apparatus

For example, in the above embodiments, the respective processing functions performed by the extraction function 371, the calculation function 372, the detection function 373, and the output control function 374 that are components of the processing circuitry 37 is performed by the X-ray CT apparatus 1 has been explained, but embodiments are not limited thereto. For example, the respective processing functions described above can be performed by a medical image-processing apparatus such as a workstation.

### X-ray Diagnostic Apparatus

Furthermore, for example, the respective processing functions can be performed by an X-ray diagnostic apparatus that can capture temporal three-dimensional medical-image data. For example, with an X-ray diagnostic apparatus that is capable of capturing temporal volume data by irradiating X-rays to the subject P from three directions different from each other, the above processing functions can be performed using the captured temporal volume data.

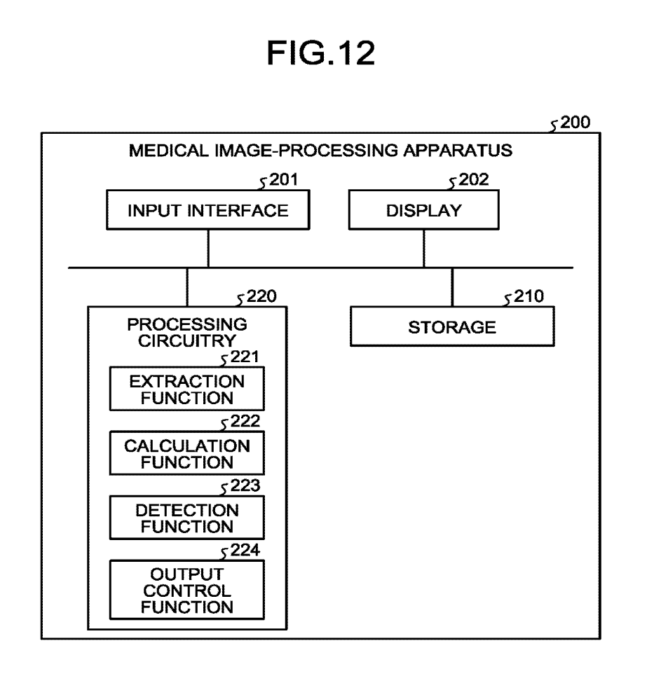

FIG. 12 is a block diagram illustrating a configuration example of a medical image-processing apparatus 200 according to another embodiment. The medical image-processing apparatus 200 corresponds to, for example, an information processing apparatus, such as a personal computer and a workstation, or an operation terminal of a medical diagnostic-imaging apparatus, such as a console device included in an X-ray CT apparatus.

As illustrated in FIG. 12, the medical image-processing apparatus 200 includes an input interface 201, a display 202, storage 210, and processing circuitry 220. The input interface 201, the display 202, the storage 210, and the processing circuitry 220 are connected so as to be able to communicate with each other.

The input interface 201 is an input device, such as a mouse, a keyboard, and a touch panel, to accept various kinds of instructions and setting requests from an operator. The display 202 is a display device that displays a medical image, or displays a GUI for an operator to input various setting requests by using the input interface 201.

The storage 210 is, for example, a NAND (not AND) flash memory or a hard disk drive (HDD), and stores various kinds of programs to display medical image data or a GUI, and information that is used by the programs.

The processing circuitry 220 is an electronic device (processor) that controls the entire processing in the medical image-processing apparatus 200. The processing circuitry 220 performs an extraction function 221, a calculation function 222, a detection function 223, and an output control function 224. The respective processing functions performed by the processing circuitry 220 are stored in the storage 210 in a form of program that can be executed by a computer. The processing circuitry 220 reads each program, and executes the read program, thereby implementing a function corresponding to each program. The extraction function 221, the calculation function 222, the detection function 223, and the output control function 224 can perform basically the same processing as the extraction function 371, the calculation function 372, the detection function 373, and the output control function 374 illustrated in FIG. 1.

For example, the extraction function 221 extracts plural regions that correspond to at least one of a lobe and a subsegment forming a lung from three-dimensional medical-image data that is imaged temporal temporally. Moreover, the calculation function 372 calculates a physical index relating to the respiratory activity for each of the extracted regions. Furthermore, the detection function 223 detects an abnormal region having an abnormality in the respiratory activity out of the plural regions by comparing temporal changes of the physical index of the respective regions. Furthermore, the output control function 224 outputs information indicating about the abnormal region. Thus, the medical image-processing apparatus 200 can detect a region of a lung having an abnormality in the respiratory activity accurately.

### X-Ray Diagnostic Apparatus

Moreover, for example, the processing functions described above can be performed by an X-ray diagnostic apparatus that can capture temporal three-dimensional medical-image data. For example, with an X-ray diagnostic apparatus that is capable of capturing temporal volume data by irradiating X-rays to the subject P from three directions different from each other, the above processing functions can be performed using the captured temporal volume data.

### Providing as Service on Network

Furthermore, for example, the processing functions described above can be provided as an information processing service (cloud service) through a network.

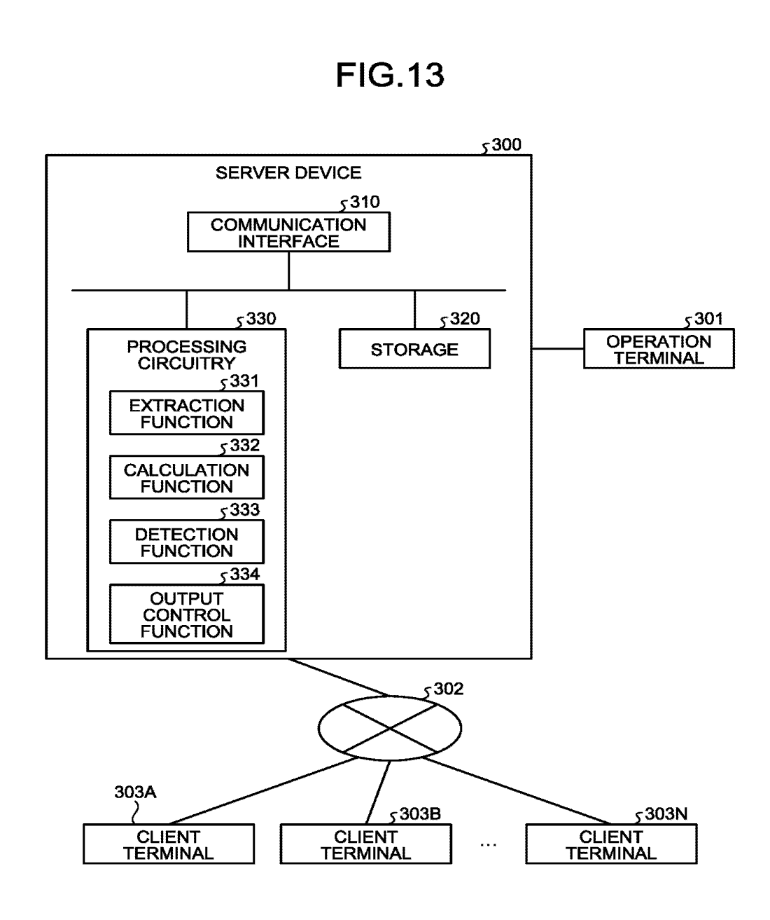

FIG. 13 is a block diagram illustrating a configuration example of a server device that provides an information processing service according to another embodiment. As illustrated in FIG. 13, for example, in a service center that provides the information providing service, a server device 300 is installed. The server device 300 is connected to an operation terminal 301. Moreover, the server device 300 is connected multiple client terminals 303A, 303B, . . . , 303N through a network 302. The server device 300 and the operation terminal 301 can be connected through the network 302. When the client terminals 303A, 303B, . . . , 303N are collectively termed without distinguishing each, it is described as “client terminal 303”.

The operation terminal 301 is an information processing terminal that is used by a person that operates the server device 300 (operator). For example, the operation terminal 301 has an input device, such as a mouse, a keyboard, and a touch panel, to accept various kinds of instructions and setting requests from an operator. Furthermore, the operation terminal 301 has a display device that displays an image or a GUI for an operator to input various setting requests by using the input device. The operator can send various kinds of instructions and setting requests to the server device 300, or can browse information inside the server device 300 by operating the operation terminal 301. Moreover, the network 302 is an arbitrary communication network, such as the Internet, a wider area network (WAN), and a local area network (LAN).

The client terminal 303 is an information processing terminal that is operated by a user that uses the information processing service. The user is, for example, a person involved in a medical field, such as a doctor and a technician that work at a medical institution. For example, the client terminal 303 corresponds to an information processing apparatus, such as a personal computer and a workstation, or an operation terminal of a medical diagnostic-imaging apparatus, such as a console device included in an X-ray CT apparatus. The client terminal 303 has a client function that can use an information processing service provided by the server device 300. The client function is recorded in the client terminal 303 in advance in a form of program that can be executed by a computer.

The server device 300 includes a communication interface 310, storage 320, and processing circuitry 330. The communication interface 310, the storage 320, and the processing circuitry 330 are connected so as to be able to communicate with each other.

The communication interface 310 is, for example, a network card or a network adapter. The communication interface 310 enables information communication between the server device 300 and an external device by connecting to the network 302.

The storage 320 is, for example, a NAND flash memory or an HDD, and stores various kinds of programs to display medical image data or a GUI, and information that is used by the programs.

The processing circuitry 330 is an electronic device (processor) that controls the entire processing in the server device 300. The processing circuitry 330 performs an extraction function 331, a calculation function 332, a detection function 333, and an output control function 334. The respective processing functions performed by the processing circuitry 330 are stored in the storage 320, for example, in a form of program that can be executed by a computer. The processing circuitry 330 reads each program, and executes the read program, thereby implementing a function corresponding to the program. The extraction function 331, the calculation function 332, the detection function 333, and the output control function 334 can perform basically the same processing as the extraction function 371, the calculation function 372, the detection function 373, and the output control function 374 illustrated in FIG. 1.

For example, a user operates the client terminal 303 to input an instruction indicating transmission (upload) of three-dimensional medical-image data to the server device 300 in the service center. When this instruction is input, the client terminal 303 transmits three-dimensional medical-image data to the server device 300. This three-dimensional medical-image data is volume data (4D CT image data) that is obtained by temporally imaging a region including a lung of a subject.

The server device receives the three-dimensional medical-image data transmitted from the client terminal 303. In the server device 300, the extraction function 331 extracts plural. regions corresponding to at least one of a lobe and a subsegment forming the lung from the three-dimensional medical-image data acquired in temporal order. Moreover, the calculation function 332 calculates a physical index relating to the respiratory activity for each of the extracted regions. Furthermore, the detection function 333 detects an abnormal region having an abnormality relating to the respiratory activity out of the plural regions by comparing temporal changes of the physical index of the respective regions. Moreover, the output control function 334 outputs information indicating about the abnormal region. Specifically, the output control function 334 transmits (causes to download) the information indicating about the abnormal region to the client terminal 303. Thus, the user of the client terminal 303 can browse, for example, the information in which a region of a lung having an abnormality in the respiratory activity is detected accurately.

That is, the processing according to the above embodiment can be provided as a medical image-processing method. The medical image-processing method includes extracting, by the server device 300, plural regions that correspond to at least one of a lobe and a subsegment forming a lung from three-dimensional medical-image data that is captured in temporal order. The medical image-processing method includes calculating a physical index relating to the respiratory activity for each of the extracted regions by the server device 300. The medical image-processing method includes detecting an abnormal region having an abnormality relating to the respiratory activity out of the plural regions by comparing temporal changes of the physical index of the respective regions by the server device 300. The medical image-processing method includes outputting information that indicates about the abnormal region by the server device 300.

Furthermore, all or a part of the processing explained as to be performed automatically out of the respective processing explained in the above embodiments and modifications can be performed manually also, while all or a part of the processing explained as to be performed manually can be performed automatically also by a publicly-known method. In addition, the processing procedures, the control procedures, the specific names and the information including various kinds of data and parameters indicated in the above document and drawings can be arbitrarily modified unless otherwise specified.

Moreover, the medical image-processing method explained in the above embodiments and modifications can be implemented by executing a medical image-processing program that has been prepared in advance by a computer such as a personal computer and a workstation. This medical image-processing method can be distributed through a network such as the Internet. Furthermore, this medical image-processing method can be stored in a computer-readable recording medium, such as a hard disk, a flexible disk (FD), a compact-disk read-only memory (CD-ROM), a magneto optical disk (MO), and a digital versatile disk (DVD), and can be executed by being read by a computer from the recording medium.

According to at least one of the embodiments explained above, a region of a lung having an abnormality in the respiratory activity can be detected accurately.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A medical image-processing apparatus comprising processing circuitry configured to:
extract a plurality of regions corresponding to at least one of a lobe and a subsegment forming a lung from three-dimensional medical-image data that is acquired by imaging in temporal order;
calculate a physical index relating to respiratory activity for each of the extracted regions;
detect an abnormal region having an abnormality relating to the respiratory activity out of the regions by comparing temporal changes of the physical index of the respective regions; and
output information indicating about the abnormal region.

2. The medical image-processing apparatus according to claim 1, wherein
the processing circuitry detects a region in which a tendency of a curve in the temporal change is different, as the abnormal region.

3. The medical image-processing apparatus according to claim 1, wherein
the processing circuitry detects a region in which an evaluation value based on a difference between a maximum inhalation point and a maximum exhalation point in the temporal change is lower than that of the other regions, as the abnormal region.

4. The medical image-processing apparatus according to claim 1, wherein
the processing circuitry detects a region in which a differential coefficient of a period including a maximum exhalation point in the temporal change is smaller than that of the other regions, as the abnormal region.

5. The medical image-processing apparatus according to claim 1, wherein
the processing circuitry detects the abnormal region by comparing a pair of regions corresponding to each other in left and right lungs.

6. The medical image-processing apparatus according to claim 1, wherein
the processing circuitry detects the abnormal region by comparing a reference region to be a reference out of the regions, with each of the regions.

7. The medical image-processing apparatus according to claim 1, wherein
the processing circuitry detects a region in which a value of a predetermined time phase in a curve of the temporal change is smaller than a value of the predetermined time phase when the value changes according to a sine curve, as the abnormal region

8. The medical image-processing apparatus according to claim 1, wherein
the processing circuitry displays a display image based on the three-dimensional medical-image data of a time phase when the abnormal region is detected.

9. The medical image-processing apparatus according to claim 8, wherein
the processing circuitry displays the abnormal region in an emphasized manner in the display image.

10. The medical image-processing apparatus according to claim 1, wherein
the processing circuitry calculates at least one of a volume, a surface area, a specific surface area, and a computed tomography (CT) value of the respective regions, as the physical index.

11. The medical image-processing apparatus according to claim 1, wherein
the processing circuitry displays a normal graph that shows a temporal change of the physical index when respiratory activity is normal in the abnormal region.

12. The medical image-processing apparatus according to claim 11, wherein
the processing circuitry generates the normal graph by changing a waveform of a template of the normal graph based on a volume of the abnormal region, and a time phase of a different region from the abnormal region out of the regions, and displays the generated normal graph.

13. The medical image-processing apparatus according to claim 1, wherein
the processing circuitry extracts a plurality of bronchus regions that correspond to a bronchus to supply air to the respective regions, and calculates the physical index for each of the extracted bronchus regions.

14. The medical image-processing apparatus according to claim 13, wherein
the processing circuitry detects either one of the abnormal region and an abnormal bronchus region having an abnormality in the respiratory activity, by comparing sets of the region and the bronchus region in correspondence.

15. The medical image-processing apparatus according to claim 13, wherein
the processing circuitry displays an image of the region and an image of the bronchus region to supply air to the region at the same time.

16. The medical image-processing apparatus according to claim 13, wherein
the processing circuitry displays a graph of temporal change of the region and a graph of temporal change of the bronchus region to supply air to the region at the same time.

17. The medical image-processing apparatus according to claim 13, wherein
the processing circuitry calculates a cross-sectional area of each of the bronchus regions also as the physical index of the bronchus region.

18. An X-ray CT apparatus comprising:
an X-ray tube configured to irradiate an X-ray to a subject;
an X-ray detector configured to detect an X-ray that has passed through the subject;
image reconstructing circuitry configured to reconstruct three-dimensional medical-image data in temporal order based on detection data of X-rays detected by the X-ray detector; and
processing circuitry configured to:
extract a plurality of regions corresponding to at least one of a lobe and a subsegment forming a lung from the three-dimensional medical-image data;
calculate a physical index relating to respiratory activity for each of the extracted regions;
detect an abnormal region having an abnormality relating to the respiratory activity out of the regions by comparing a temporal change of the physical index of the respective regions; and
output information indicating about the abnormal region.

19. A medical image-processing method comprising:
extracting a plurality of regions corresponding to at least one of a lobe and a subsegment forming a lung from three-dimensional medical-image data that is acquired by imaging in temporal order;
calculating a physical index relating to respiratory activity for each of the extracted regions;
detecting an abnormal region having an abnormality relating to the respiratory activity out of the regions by comparing temporal changes of the physical index of the respective regions; and
outputting information indicating about the abnormal region.
